(19) United States
(12) Patent Application Publication
(10) Pub. No.: US 2020/0193798 A1
(43) Pub. Date: Jun. 18, 2020

(54) HYGIENE COMPLIANCE MONITORING

(71) Applicant: Essity Hygiene and Health Aktiebolag, Goteborg (SE)

(72) Inventor: Hakan Lindstrom, Goteborg (SE)

(21) Appl. No.: 16/607,859

(22) PCT Filed: Apr. 27, 2017
(86) PCT No.: PCT/EP2017/060065

§ 371 Date: Oct. 24, 2019

(51) Int. Cl. G08B 21/24; G09B 19/00; G06Q 10/06; G16H 40/20; G06K 7/10

(52) U.S. Cl. CPC G08B 21/245; G09B 19/0076; G06K 7/10366; G06Q 10/06393; G16H 40/20; G09B 19/003

(57) ABSTRACT

A system is provided for determining a hygiene compliance metric which indicates a usage of hygiene equipment. The system includes distributed hygiene equipment arranged to dispense a hygiene consumable and/or to dispose of a hygiene consumable; a tag configured to transmit and/or receive radio signals; positioning equipment arranged to determine information on a position of said tag by receiving and/or transmitting radio signals from and/or to said tag; and a processing entity. The processing entity is configured to receive said information on a position of said tag, to define a zone into which said tag may enter, to define a rule, and to calculate said hygiene compliance metric based on said information on the position, said zone, and said rule.

### CROSS-REFERENCE TO RELATED APPLICATION

This application is a national phase entry of, and claims priority to, International Application No. PCT/EP2017/060065, filed Apr. 27, 2017, with the same title as listed above. The above-mentioned patent application is incorporated herein by reference in its entirety.

### TECHNICAL FIELD

This application generally relates to determining a compliance indicator or metric in the context of hygiene equipment, such as soap, disinfectant, towel dispensers, and the like. For example, the application relates to determining a hygiene compliance indicator by tracking the use of hygiene equipment and observing movement and/or behavior of individuals (users) who are supposed to use hygiene equipment at specific instances and/or according to applicable rules.

### BACKGROUND

Hygiene equipment is commonplace today in many facilities, such as hospitals, medical service centers, intensive care units, day clinics, private practices, lavatories, rest rooms, hotels, restaurants, cafes, food service places, schools, kindergartens, manufacturing sites, administration and office buildings, and, in general, places and facilities that are accessible to the public or to a considerable number of individuals. The mentioned hygiene equipment thereby includes various types of individual devices and installations such as soap dispensers, dispensers for disinfectant solutions, gels or substances, towel dispensers, glove dispensers, tissue dispensers, hand dryers, sinks, radiation assisted disinfectant points, ultraviolet (UV) light, and the like.

Although such hygiene equipment is commonplace today in many places, the use thereof by the individuals visiting these places or working in these places is still oftentimes not satisfactory. For example, hospitals, and, in general, medical service centers often suffer from hygiene deficiencies, which, in turn, may lead to the spread of infections and related diseases. In particular, such insufficient hygiene amongst medical care personnel coming into close contact with patients and bodily fluids can lead to the spread of infectious diseases amongst the personnel and other patients. It is also known that infections by highly resistant bacteria pose a severe problem in such places, especially in hospitals. In general, so-called Healthcare Associated Infections (HAI) are a real and tangible global problem in today's healthcare. HAI can be found to be currently the primary cause of death for 140,000 patients/year, affecting millions more and costs society in the range of billions of dollars per year.

At the same time, however, it is known that hygiene, and, in particular, hand hygiene, is an important factor as far as the spread of infectious diseases are concerned. Specifically, medical care personnel should make proper use of hand hygiene as often as possible so that the spread of bacteria and other disease causing substances is minimized. The actual usage of such hygiene equipment, however, may depend on—amongst others—the management of the facility, accessibility and usability of the equipment, culture, the cooperation and will exercised by the individuals working in these places or visiting such places, training of individuals, time pressure and possibly also other factors. In other words, an important factor remains the fact that individuals may not make use of installed and provided hygiene equipment although they are supposed to. Furthermore, it is generally accepted that an increased use of hygiene equipment can substantially contribute in reducing the spread of bacteria and the like, which, in turn, can drastically reduce the appearance of related infections and diseases.

For example, a corresponding relatively “low” compliance may indicate that the actual use of hygiene equipment is not satisfactory, whilst relatively “high” compliance may indicate that the actual use of hygiene equipment corresponds, within a given threshold, to some target usage, and, consequently, may be regarded as being satisfactory. A tangible figure for estimating the quality of hygiene compliance may be found in a so-called compliance metric that as such may provide many advantages, since it can give a concise picture to operators of the corresponding facility so that they may initiate measures for increasing and promoting the actual use of hygiene equipment.

Therefore, there are already ways of measuring and/or estimating such a compliance metric in the arts, wherein the conventional approaches usually rely on measuring and/or observe “manually” by a human observer so-called opportunities and comparing these obtained opportunities to a measured/detected/observed actual use of the hygiene equipment. In other words, the opportunities indicate any event when hygiene equipment should or could have been used. By then comparing this “should/could”-value to an actual usage value a compliance metric can be calculated, as e.g. a percentage value or a ratio. In general, the opportunities can be well defined figures, since they may be associated to specific rules and/or recommendations. For example, the World Health Organization (WHO) has defined a so-called “Five Moments Of Hand Hygiene” including as explicit definitions for opportunities: 1. Before patient contact; 2. Before aseptic task; 3. After body fluid exposure risk; 4. After patient contact; and 5. After contact with patient surroundings.

Besides manually measuring opportunities and detecting the usage of hygiene equipment there exist also fully automated systems, where opportunities are detected by tags carried by an individual subject to hygiene compliance and associated equipment for detecting when a tag moves into a given vicinity as, for example, taught by U.S. Patent Publication No. 2013/0027199. The latter may define a zone, for example around a patient's bed that requires the use of hygiene equipment before entering and/or after leaving the zone. For example, having not used hygiene equipment before entering an/or leaving a zone around a patient's bed or leaving the room may indicate non-compliance, whilst having used hygiene equipment before entering the zone and/or after leaving the zone may indicate compliance.

However, the mentioned arts determine the metrics on hygiene compliance by conventional ranging, locating, and tracking techniques that are not specifically adapted to the applicable preconditions and do not take into account the characteristic circumstances of tracking devices and/or individuals in the context of hygiene compliance. In particular, technologies such as satellite based positioning systems (e.g. Global Positioning System (GPS), Galileo, GLONASS, WAAS, etc.) and mobile network locating services (via GSM, PCS, DCS, GPRS, UMTS, 3GPP, LTE, etc.) have only a limited accuracy and availability indoors, where most of the tracking may occur in the context of determining a hygiene compliance. Similarly, conventional indoor techniques, such as employing a received signal strength (RSS) provide firstly only a limited accuracy and, secondly, are susceptible to interference with other radio signal sources, including the ubiquitous mobile phone and wireless LAN networks and installations. At the same time, a sufficient accuracy can be vital for a system so that it produces a reliable figure on the hygiene compliance. Specifically, the required precision may relate to the “human scale”, i.e. in many cases an “arm-length” that usually translates to below 1 m and preferably below 50 cm.

Thus, it would be desirable to provide improved concepts of determining a hygiene compliance in connection with the actual and appropriate use of distributed hygiene equipment. Equally there is a need for improved equipment that determines a hygiene compliance and conveys corresponding information to appropriate addressees for ultimately encouraging the actual use hygiene equipment.

### SUMMARY

To address these and other problems with conventional designs, according to an embodiment of the present invention there is provided a system for determining a hygiene compliance metric which indicates a usage of hygiene equipment. The system includes distributed hygiene equipment arranged to dispense a hygiene consumable and/or to dispose of a hygiene consumable and a tag configured to transmit and/or receive radio signals. The system further includes positioning equipment arranged to determine information on a position of the tag by receiving and/or transmitting radio signals from and/or to the tag, and configured to determine information indicating a time of flight of a radio signal transmitted between the tag and the positioning equipment for determining the information on a position. The system also includes a processing entity which is configured to receive the information on a position of the tag, to define a zone into which the tag may enter, to define a rule, and to calculate the hygiene compliance metric based on the information on the position, the zone, and the rule.

According to another embodiment, there is provided a method for determining a hygiene compliance metric which indicates a usage of hygiene equipment. The method includes detecting a usage instance of distributed hygiene equipment arranged to dispense a hygiene consumable and/or to dispose of a hygiene consumable; transmitting and/or receiving radio signal from and/or to a tag; determining information on a position of the tag by receiving and/or transmitting radio signals from and/or to the tag and by determining information indicating a time of flight of a radio signal transmitted between the tag and the positioning equipment; receiving the information on a position of the tag; defining a zone into which the tag may enter; defining a rule, and calculating the hygiene compliance metric based on the information on the position, the zone, and the rule.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, explain the one or more embodiments of the invention.

FIG. 1 is a schematic view of a typical environment where the actual usage of hygiene equipment is subject to compliance, which may be used with one embodiment.

FIG. 2 is a schematic view of a deployment of a system according to an embodiment of the present invention into an environment similar to the one shown in FIG. 1.

FIG. 3 is a schematic view of a processing entity used with the system according to an embodiment of the present invention.

FIG. 4A is a schematic view of a tag from a functional point of view, the tag being usable with the system according to embodiments of the present invention.

FIG. 4B is a top perspective view of a tag in the form of compact electronic device, in accordance with another embodiment.

FIG. 4C is a top perspective view of a tag in the form of a smartphone or similar device, according to another embodiment.

FIG. 5A is a schematic view of positioning equipment for determining information indicating a time of flight of a radio signal according to one embodiment.

FIG. 5B is a schematic view of positioning equipment for determining information indicating a time of flight of a radio signal according to another embodiment.

FIG. 6 is a flowchart of one embodiment of a method according to the present invention.

### DETAILED DESCRIPTION

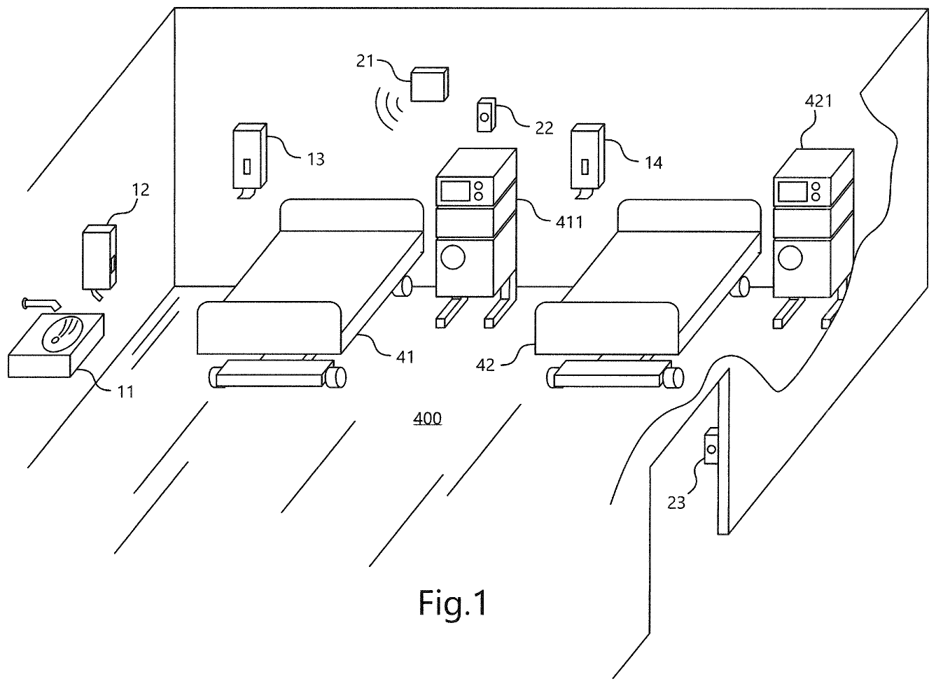

FIG. 1 shows a schematic view of a typical environment where the actual usage of hygiene equipment is subject to compliance, and where the individuals are usually encouraged to use the hygiene equipment at specific instances. As an example, there is shown as a facility an intensive care unit 400 with corresponding intensive care points: first and second patient stations 41, 42 and first and second patient care equipment 411, 421. As can be seen, the intensive care unit 400 may be occupied by one or two patients in the shown configuration, whilst the embodiments of the present invention may envisage also other intensive care units with any number of patients and personnel and/or other facilities as mentioned elsewhere in the present disclosure. Examples for other possible working environments include hospitals and medical service centers in general, day clinics, private practices, lavatories, rest rooms, hotels, restaurants, cafes, food service places, schools, kindergartens, manufacturing sites, administration and office buildings, and, in broad terms, places and facilities that are accessible to the public or to a considerable number of individuals.

The configuration shown in FIG. 1 can acquire data indicating the usage of the hygiene equipment from equipment sensor arrangement provided for or in one or more of the individual pieces of hygiene equipment, such as a washing sink 11, a soap dispenser 12, and a first and a second disinfectant dispenser 13, 14. In this way, the system is able to receive usage data from these pieces of equipment 11-14 as possibly individual signals from each corresponding device/sensor. Other examples of hygiene equipment may include a towel dispenser, an alcogel dispenser, a tissue dispenser, a hygiene article dispenser, a waste bin, a used towel bin, and a toilet paper dispenser. Likewise, opportunities can be detected by corresponding sensors including a vicinity sensor 21, a light barrier sensor 22 and a door passing sensor 23. As already mentioned earlier, the data on the usage and on the opportunities can be collected and processed for calculating a hygiene compliance metric or indicator, which, in turn, indicated to what degree the individuals (e.g. nurses, doctors, and caretakers) use the hygiene equipment at appropriate opportunities.

Normally, the data generated by the distributed equipment 11, 12, 13, 14, 21, 22, and 23 is retrieved by some kind of central data processing and storage entity (not shown, e.g. a server), where the hygiene compliance metric is calculated. However, the calculated hygiene metric can only be as accurate as are the corresponding basis data. For example, an inaccurate positioning of an individual may result in determining an opportunity for using hygiene equipment when actually no opportunity existed. As a result, the individual may be attributed wrongly with achieving only a low compliance contribution. A door passage sensor 23 by itself may not be able to monitor movement between the beds 41 42, thus not detecting potential for patient cross-contamination. Such inaccurate data acquisition may lead to an inaccurate estimation of the compliance which may then be perceived negatively by the individuals, and, eventually, such circumstances may result in a decreased acceptance of the system so that the ultimate goal of promoting the use of hygiene equipment by the individuals may be missed. Naturally, also the reverse case can happen that the calculated metric suggests good hygiene compliance while it is actually not.

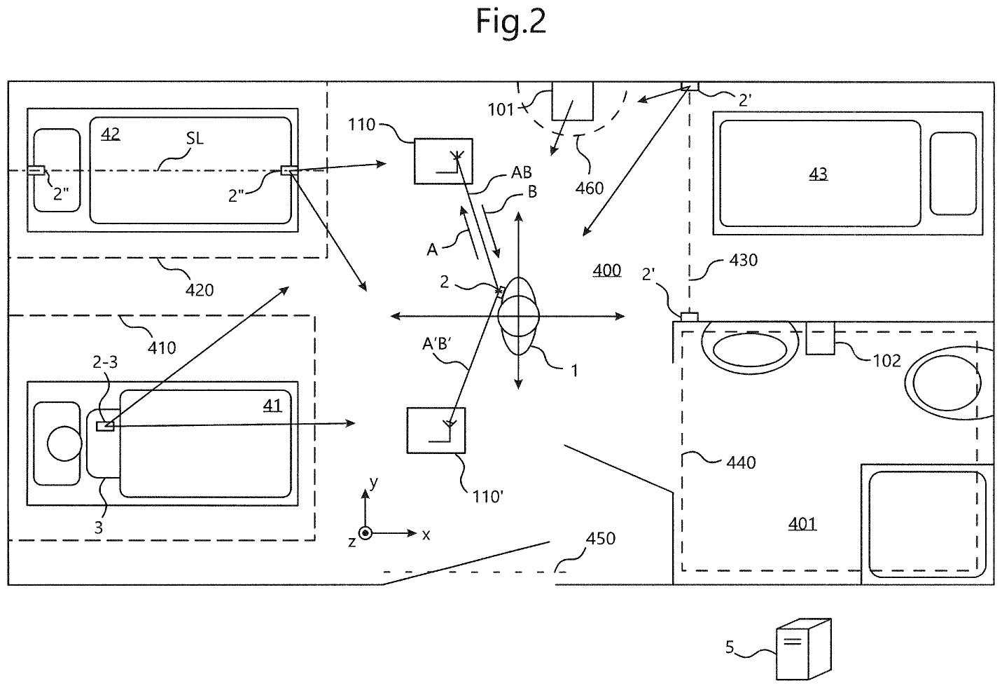

FIG. 2 shows a schematic view of a deployment of a system according to an embodiment of the present invention. Specifically, the deployment is explained again along an example of a hospital environment, which is seen from atop and comprises patient stations (beds) 41 (with patient 3), 42, and 43. An individual 1, e.g. a doctor, a nurse, a member of personnel, another patient or a visitor, can move freely in the ward 400 for fulfilling any given task(s). The individual 1 carries a tag 2 which is explained in greater detail elsewhere in the present disclosure.

For example, the individual 1 may be assumed to have visited the restroom/bathroom 401 and is now heading toward a patient lying in patient station 42. A compliance rule may require that an individual 1 uses hygiene equipment (e.g. a soap dispenser 102) before leaving a restroom such as bathroom 401 having a shower, a toilet, and a hand wash basin. A “compliant” individual 1 may wash his/her hands and may use the soap dispenser 102. This piece of hygiene equipment 102 may be configured to detect the usage instance and send out a signal over a wired or wireless interface and via some kind of network to a central data processing and storage entity (e.g. a server).

Generally, an embodiment of the present invention is a system for determining a hygiene compliance metric which indicates a usage of hygiene equipment. The system as shown may comprise firstly distributed hygiene equipment that is arranged to dispense a hygiene consumable and/or to dispose of a hygiene consumable, and that is arranged to detect a usage instance indicating a use of the hygiene equipment by a user, and to send information on the usage instance toward a processing entity. As mentioned, the individual 1 may use the soap dispenser 102 as one exemplary form of a piece of hygiene equipment, and this detected usage can be forwarded to a (remote) server 5 as a form of the processing entity. The mentioned tag 2 can be likewise part of the system and can be configured to at least transmit radio signals and can be arranged to be carried by the individual 1, i.e. a user. The tag 2, just as the individual 1, can move in the ward basically in the three dimensions x, y, and z, wherein the z-direction is assumed to coincide with the vertical direction and thus to be perpendicular to the drawing plane.

The system further comprises positioning equipment that is arranged to determine information on a position of a tag by at least receiving radio signals from it, and that is configured to determine information indicating a time of flight of a radio signal transmitted between the tag and the positioning equipment for determining the information on a position. In one embodiment, the positioning equipment is arranged to consider a line of sight AB between a piece of positioning equipment, e.g. the device 110 in FIG. 2, And tag 2. Specifically, this may involve the transmission of a radio signal in direction A, a radio signal in direction B, or two or more radio signals covering both directions A & B. Further details on the appropriate exchange of radio signals and possibly information as carried by the radio signal (s) as payload data are provided below in the present disclosure. In any way, the device 110 can receive and or transmit radio signal(s) with tag 2 and obtain information on a time of flight along the line AB. A similar procedure may be carried out with a further piece 110′ of positioning equipment with regard to the line of sight A′B′. As a result, information will be available indicating at least one distance between the tag 2 and a known position, e.g. the position of device 110.

Generally, the positioning equipment can be in the form of distributed individual devices mounted on the ceiling (as shown), mounted on the wall or incorporated into the floor, or any combination thereof. As a further option, the positioning equipment can also be in the form of devices and/or functionalities that are combined with or integrated into other devices, such as pieces of hygiene equipment, dispensers, instruments, infrastructure components, power outlets, light switches, etc.

Corresponding information can be forwarded to server 5 or also locally, i.e. in the tag 2 or the device 110 and/or 100′, processed to determine information on a position of tag 2. This processing may consider further measured or assumed information so as to determine a location of tag 2. For example, the carrying height in the z-direction may be further measured or assumed. In the latter case, this may be accomplished by affixing the tag 2 to the individual at a known height. This height may be known to the server 5 or may also be made known to tag 2 so that it can convey corresponding information as payload data to a radio signal. In this case the tag 2 may comprise means to configure the actual height and means for generating corresponding payload data and radio signals.

In general, information on one distance may be enough to determine a usage instance or an opportunity. For example a zone may be defined as a zone of a constant distance around a known point or object, as e.g. shown around dispenser 101 that acts as a further piece of positioning equipment with its associated zone 460. Entering this zone may be interpreted as that an individual carrying a tag uses the hygiene equipment. In this way, the information on the usage instance is obtained in terms of information on a position of a tag in connection with a specific zone—here zone 460 around dispenser 101. In this case it may be sufficient to determine only one distance between a tag and the dispenser 101 so as to make a determination whether the tag is inside or outside zone 460. Generally, the server 5 may receive this information and consider one or more rules that then allows it to calculate the hygiene compliance metric based on the information on the usage instance, and the information on the position in connection with at least one zone.

In general, however, information on two or more distances can be considered in order to calculate—e.g. in one of the devices 110, 110′ or in the server 5—a position of tag 2 in two or even three dimensions and/or in order to achieve an overall improved accuracy of any location/position information. Generally, also one dimension may be estimated e.g. by assuming that a tag is carried at a more or less fixed height z as, for example, a hip or breast height. This may be effected by employing so-called Kalman and/or Particle filters, other related and suitable filters, and/or multilateration which are as such known ways for determining information on a position from the basis of information indicating two or more distances to known points or positions. In this way, the system may further comprise the server 5 as a processing entity. This server can then be configured to receive the information on the usage instance, to receive the information on a position of the tag, to define a zone into which the tag may enter, to define a rule, and to calculate the hygiene compliance metric based on the information on the usage instance, information on the position, the zone, and the rule.

Specifically, the mentioned zones may be defined in terms of “virtual” zones and/or one or more lines around beds, rooms, hygiene equipment, and any other objects and positions which generally relate to a usage of hygiene equipment and/or an opportunity in relation with the use of hygiene equipment. In this way, zones 410, 420, and 430 may be defined for, respectively, beds or patient stations 41, 42, and 43. Likewise, a zone 440 may be defined for the bathroom 401, and the zones can also be determined in terms of lines 450 and 430 that may be seen as singular zone boundaries. In particular, crossing a line 450 or 430 may result in a determination of entering or leaving a zone whilst the remaining boundaries of the zones are not, or do not need to be defined (e.g. like in the case of the patient station 43 in the corner of room 400). In any way, such zones may define opportunities, in the sense that a tag 2 entering such a zone indicates to the system that the individual carrying tag 2 was supposed or is now supposed to use hygiene equipment.

As far as the zones as such is concerned, different schemes may be adopted for their respective definition. Firstly, a zone may be a purely “virtual” zone such as zone 440, which can be defined by respective information, for example, in the server 5. In this way, the processing of server 5 may determine whether a tag 2 enters or leaves the zone 440 by receiving information from the positioning device 110/110′ and calculating a position of tag 2 in relation to the zone 440. Likewise, a zone may also be defined “locally” and/or dynamically by respective tags 2′, 2″, and 2-3.

For example, tags 2′ may be configured to define corners of a zone or borderline. As can be seen in FIG. 2, the system may define a zone or borderline 430 by locating tags 2′ with device 110 or 110′. The radio signals emitted from tags 2′ may carry payload which can identify their respective association to one another and/or information on a type of zone (e.g. borderline, rectangle zone with dimensions and further positional data/definitions relative to the tags' positions). Similarly, tags 2″ may be arranged along a symmetry line SL of bed 42. According information may be conveyed toward or may be present in server 5 so as to define zone 420.

A further embodiment considers the definition of a moving or dynamic zone. For example, patient 3 is provided with a tag 2-3 that defines a zone 410 around patient 3 regardless of his/her current position. This allows determining correct compliance regardless of whether the target is mobile and is thus able to move outside to an otherwise fixedly defined zone. Likewise, a tag 2-3 could also be affixed to a bed, to a specific point in a room or to a piece of equipment (medical devices, cleaning trolley, etc.). In all, the definition of zones by tags may provide the advantages of easy definition of zones, dynamic definition of zones, easy modification and deactivation/activation of zones. Different tags may also indicate zones with different characteristics such as geometrical shape of the zone, compliance rules to be used for the particular zone, etc. For example, tags can be provided with a simple user interface (switch, sensor, LED, LCD, etc.) so as to allow an on-the-spot activation/deactivation and/or setup of characteristics. Also, the characteristics may be configured remotely. The system may thus in some embodiments be able to consider zones only when they are actually “hot” in the sense that—for example—a bed is indeed occupied.

In general, it is noted that the positioning equipment can be implemented as part of a tag itself or separate therefrom as, for example, shown as devise 101, 110, 110′. Specifically, the information on the position may be determined between two tags alone, or one or two tags and devices 110, 110′, . . . . In the case of device 101 or tag 2-3 it may not be necessary to involve devices 110 or 110′, since the determination of a distance between two involved points may be sufficient to determine a position relative to a zone. It may be fully sufficient to calculate a value that indicates “in the zone” or “not in the zone”, and any additional positional information or precision may be superfluous.

Any zone can be generally defined on a “human scale” taking the measurements of the human body as the reference. A width of a zone around a bed may therefore be slightly more than arm's length, so as to be in the range of 50 cm to 100 cm, implying that a person outside this zone can be assumed to have no chance of touching the patient in the bed whereas the same person inside the zone at least has a theoretical possibility to touch the patient and is therefore subject to hand hygiene regulations, and, in turn, hand hygiene compliance observation.

The individual 1 may then proceed toward patient station 42. A further compliance rule may require that individual 1 uses hygiene equipment (e.g. a disinfectant dispenser 101) before coming into contact with the patient at station 42. Here, the determination of the usage and the opportunity may be vicinity or location based in connection with zone 460. Similarly, the (corresponding) opportunity may be determined based on a location relative to the target, in this case the patient station 42. Specifically, server 5 may define the zone 420 and consider a tag entering the zone 420 as an instance of an opportunity to now use or to have briefly before used hygiene equipment. This determination may be simply based on the assumption that an individual carrying tag 2 and entering zone 420 will also get in close or physical contact with the patient and/or the surroundings.

This is the very reason, why an opportunity is associated with a usage instance, since the physical contact to patient should be preceded by using hygiene equipment in order to minimize the spread of potentially infectious bacteria, viruses, fungi, etc. Likewise, also the physical contact with a patient may be associated with a usage of hygiene equipment thereafter in order not carry anything infectious from that patient to others. Having the information on a hygiene compliance metric at hand may now allow a feedback sequence for conveying information on the achieved hygiene compliance to the users/individuals. For this purpose, the server 5 may be employed to retrieve and analyze the information and to take any suitable and desired feedback actions so that the use of hygiene equipment by the individuals is actually encouraged.

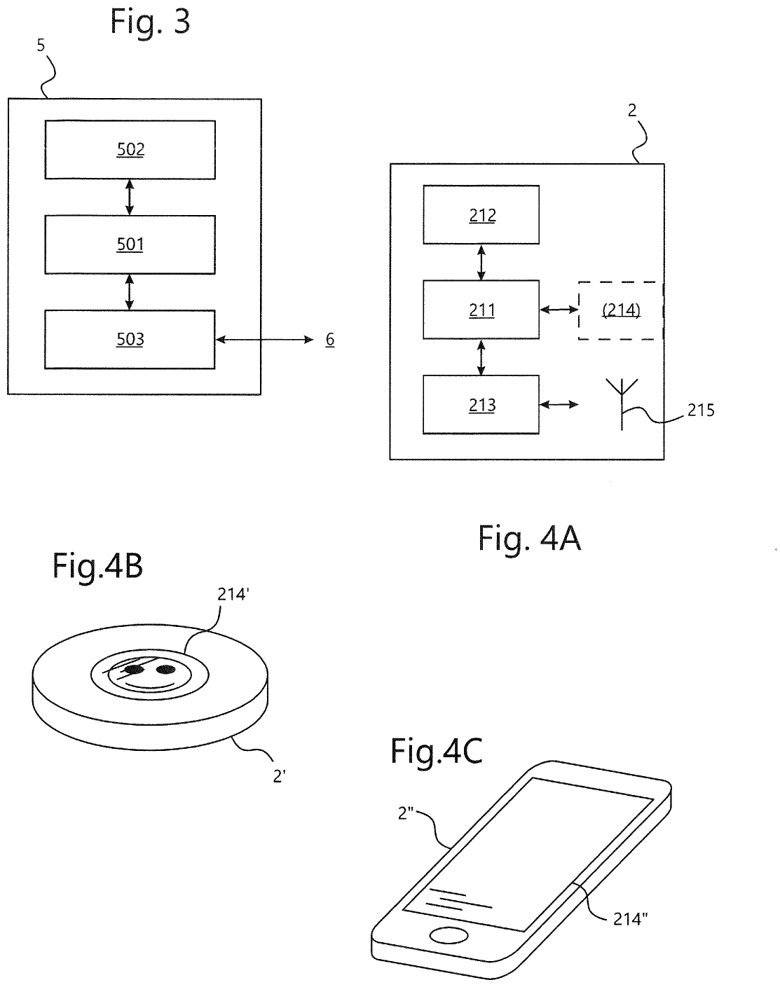

FIG. 3 shows a schematic view of a processing entity according to an embodiment of the present invention. Specifically, the processing entity 5 can be in the form of a server or personal computer, or, more generally, in the form of processing resources of a cluster or datacenter. The processing entity 5 may be part of a system for determining a hygiene compliance metric which indicates a usage of hygiene equipment. For this purpose, the server entity 5 may comprise or may have access to processing resources 501, memory resources 502 and communication resources 503, where the latter establish a communication path via one or more networks 6 toward distributed hygiene equipment, a tag, and/or positioning equipment. In this way, entity 5 can receive information on a usage instance from the hygiene equipment, determine information indicating a time of flight of a radio signal transmitted between the tag and the positioning equipment for determining the information on a position of the tag.

This and other functionalities may be implemented as code stored in the memory resources 502 that can instruct the processing resources (or circuit) to receive and process the data in connection with information on the usage instance and with information on the position of a tag. The code may further implement a definition of one or more zone into which the tag my enter, to define one or more rules, and to calculate the hygiene compliance metric based on the information on the usage instance, information on the position, the zone, and the rule.

As regards the mentioned rules, a determination of an opportunity (e.g. entering of a bed zone) without a prior usage determination will indicate non-compliance, just as an elapse of a time span after a determined opportunity (e.g. entering of a room) without an associated usage determined. In general, a determined usage or opportunity may be internally handled by the processing resources 501 as a data record carrying type and association information. For example the association information of an opportunity record can store information on an associated type of usage record. If a corresponding pair is determined within a corresponding time span the processing resources 501 may determine compliance and accordingly set an compliance indicator or a contribution to a metric to a corresponding value, e.g. “1”. Likewise, if no corresponding pair is determined within a time span the processing unit 211 may determine non-compliance and accordingly set the indicator/contribution to a corresponding non-compliance value, e.g. “0”.

An alternative mechanism would involve the storing and processing of vector in the form of, for example, [usage opportunity], where a compliance indicator indicating compliance could be obtained for [1 1], whereas a compliance indicator indicating non-compliance could be obtained for [1 0] or [0 1]. This mechanism may add flexibility in analysis as well as the possibility to also consider metadata in an easy manner. For example, additional values x, y, . . . may be considered as [usage compliance x y . . . ] for expressing compliance to additional rules relating to, for example, a time, a position, a user ID, and the like. In general, however, the mentioned metadata may naturally also be considered by the above mentioned data records in the form of additional fields.

FIGS. 4A to 4C show schematic views of deployments of tags according to respective embodiments of the present invention. FIG. 4A shows a schematic of a tag 2 from a functional point of view. The tag 2 is generally adapted to be carried by the user and comprises a radio unit 213 that is configured to at least transmit radio signals via an antenna 215, and a processing unit 211, and—optionally—an operation/notification unit 214. The tag 2 may further comprise a memory unit 212 that may store code for instruction the processing unit 211 to implement any desired functionality. However, the configuration may well be integrated into the processing unit 211 itself, so that no separate or individual memory unit 212 is necessary. The radio unit 213 may employ any suitable technology and protocols and preferred technologies include Bluetooth, WiFi, WLAN, WiMAX, UWB (Ultra wide band), and the like.

The processing unit 211 is generally configured, for example by respective programming, to instruct the radio unit 213 to transmit radio signals that can be received by positioning equipment in order to determine information on a position of tag 2. Further, the operation/notification unit 214 may be employed to operate the tag as such (e.g. configuration, set and modify settings, etc.) or to convey information to the user in accordance with the determined hygiene compliance. In this way, it is possible to provide an individual immediately with a feedback on a good compliance or non-satisfactory behavior. Furthermore, the tag 2 can assist in collecting any desired information and can even also carry out positioning by determining and processing information on a time of flight of a radio signal exchanged between the tag and another entity (see also the description in conjunction with FIG. 2).

FIG. 4B shows a schematic of a tag 2′ in the form of compact electronic device. The tag 2′ will internally comprise all the necessary functional features as described above in conjunction with FIG. 4A. In this embodiment, however, the operation/notification unit at least comprises a display 214′, optionally a touch-sensitive display, which can be instructed also, for example, to display a positive emoticon in case of determining compliance. A negative emoticon may be displayed for conveying a non-compliance to the user. This display may be accompanied by any acoustic and/or vibrational signal in order to make the user aware of the feedback also in situations where the tag 2′ is carried inside a pocket or not in the immediate visible range of the user (e.g. attached to a shirt).

FIG. 4C shows a schematic of a tag 2″ in the form of a smartphone or similar electronic device. The device defining the tag 2″ will internally comprise all the necessary functional features as described above in conjunction with FIG. 4A. In this embodiment, however, the operation/notification unit at least comprises the device's display 214″ which can be instructed to display any content for indicating compliance and non-compliance. It should be clear that the functionalities may be implemented by a program or application (“app”) which instructs the device's resources as a form of radio unit, a notification unit, and a processing unit. Again, any display may be accompanied by any acoustic and/or vibrational signal in order to make the user aware of the feedback also in situations where the device is outside the immediate visible range for the user.

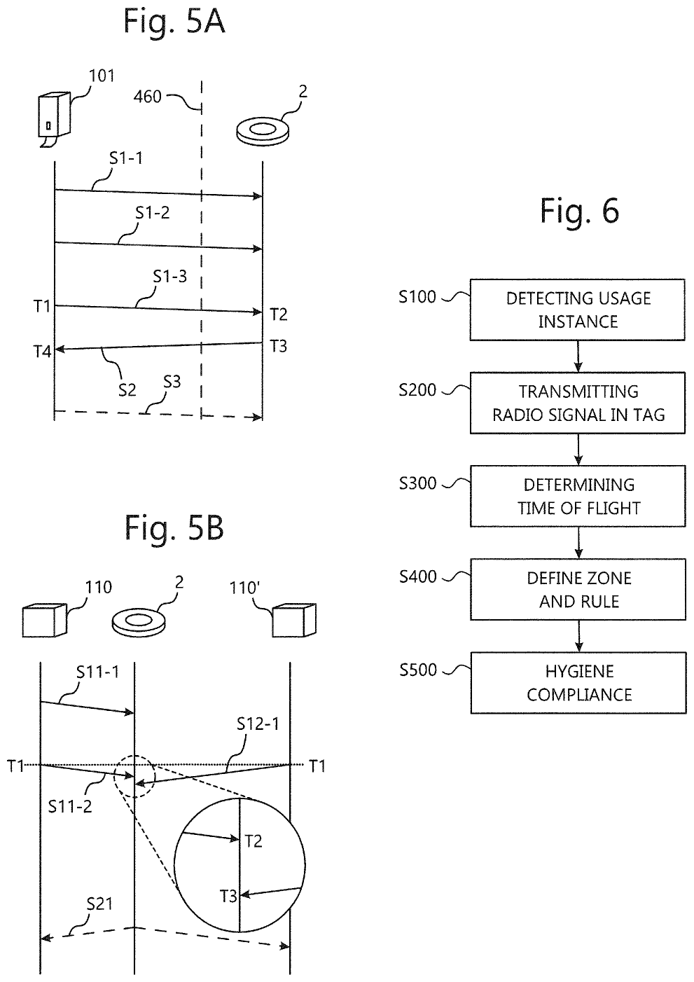

FIG. 5A shows a schematic view of positioning equipment for determining information indicating a time of flight of a radio signal transmitted between a tag and positioning equipment for determining information on a position. Specifically, it is shown a schematic view of a mechanism for ranging by employing a time-of-flight determination of radio signals according to an embodiment of the present invention. In a way, FIG. 5A shows schematically a general variant of so-called two way ranging (TWR) between a piece of positioning equipment (here the piece of hygiene equipment 101, but generally any suitable beacon or positioning device) and a tag 2. It is thus assumed that the device 101 transmits beacon signals S1-1, S1-2, in regular or irregular intervals. At some given time, the tag 2 comes into range and can receive the beacon signal S1-3. The tag 2 can obtain information on the timing when the signal S1-3 was emitted (T1) and received (T2) and obtain information on when a response signal S2 is transmitted (T3) toward and received (T4) at the device 101. Optionally, a third signal S3 can be employed to convey all the required information and data to tag 2.

A payload in the beacon signal S1 may contain information on identifying the device 101, and this information may be encoded, together with relative or absolute information on the receiving/transmission timings, onto the payload of the response signal S2. The dispenser can thus obtain information on the timing when the signal S2 was received (T4) and obtain information on the timing when the signal S1-3 was transmitted (T1). Together with the timing information contained in payload data of signal S2, the processing unit of the tag 2 is able to determine the distance d between the device 101 and the tag 2 by employing a calculation such as

$$d = c \times \frac{[ ( T 4 - T 1 ) - ( T 3 - T 2 ) ]}{2} , \qquad ( 1 )$$

where c denotes the speed of light, the applicable propagation speed for radio signals. Further, the conveyed payload may also be employed to ensure that the signal S2 is in actual response to the beacon signal S1-3. In addition to this, further signals may be employed for any one of improving accuracy, employing cancelling techniques or adding redundancy. In any way, the tag 2 can compile at least a distance relative to the device 101 for determining whether or not a zone 460 has been entered and/or left. If one or more additional distance(s) to another or other device(s), or one or more additional distance to device 101 (e.g. with respect to a second and further antenna thereof) is obtained, multiple distances are available for also compiling information on a relative position in two or even three dimensions.

It should be clear that now information is available that allows the determination indicating a position in relation to a zone, at least to the extent of “in zone” or “out of zone”, for example in relation to a distance-defined zone 460. The same or similar message sequence may be employed between tag 2 and the devices 110 and 110′ as further explained in conjunction with FIG. 2. In this case at least two distances would be available that already allow the calculation of a position in two dimensions. Such information can then be evaluated for example in the server 5 in relation to a zone defined therein. In particular, it can be determined whether tag 2 leaves zone 440 for registering an associated opportunity instance. Likewise, the position of tag 2″ or 2-3 could be determined in a similar manner, and the server 5 may evaluate the position of tag 2 in relation to zones 420, 410 that are (dynamically) defined by the positions determined for tags 2″ or 2-3.

A similar ranging scheme can be employed where it is assumed that the tag 2 transmits beacon signals S1-1, S1-2, in regular or irregular intervals. The ranging is carried out similar to the situation of FIG. 5A, taking into account—at least indirectly—the timings T1 to T4. An additional signal may be employed if the distance determination is made at the site of the device 101 but information on the determined distance should be conveyed back to the tag 2.

FIG. 5B shows a schematic view of another principle way to obtain information on a position using similar technology in the context of the so-called time difference of arrival (TDOA) scheme, which usually involves more than one device and the tag 2. Specifically, two pieces 110 and 110′ of positioning equipment represent beacon devices and transmit beacon signals S11-1, . . . and, respectively, S12-1, . . . into some overlapping range. The signals may be synchronized in time or may at least have some known timely relationship. At some point in time, the tag 2 is assumed to have received the two beacon signals S11-2 and S12-1. Both devices 110 and 110′ obtain information on timing when the signals S11-1 and S12-1 are transmitted by their respectively coupled antennae. In this embodiment, the information on the timing can be identified as an instruction or synchronization signal employed for the plurality of devices 110 and 110′ to transmit the signals S11-1 and S12-1 at substantially the same time T1. If the beacon signals can be assumed to be emitted in a synchronized fashion, then it may suffice to focus on the time difference of arrival at tag 2 for calculating the desired information on a position.

In this way, the tag 2 may determine different timings when the different signals are received. Namely, the signal S11-2 can be assumed to be received at T2 at tag 2, and the signal S12-1 can be assumed to be received at T3 at tag 2. With this knowledge, the tag 2 can initiate ranging calculations. Again, further signals may be employed for any one of improving accuracy, employing cancelling techniques or adding redundancy. In addition, any determined distance or difference may be conveyed via an optional signal S21 to any one of the involved devices.

Similarly to the one described in conjunction with FIG. 5B, the tag 2 can be the originator of the beacon signal. Therefore, also the tag 2 can be assumed to transmit the beacon signals at regular or irregular intervals. It may now be assumed that device 110 receives a particular signal at time T2, whereas device 110′ receives this particular signal at time T3. Again, the payload carried by the signal may be employed for facilitating identification and association of any received signals. The devices can obtain information on the receiving times T2 and T3 and can decode any payload to accomplish the mentioned association, so as to determine a time difference of arrival of one signal at different locations. This information may be fed back to the tag 2. As for further possible ways of initiating the sequence, it is noted that the configuration shown in the FIGS. 5A and 5B can be modified so that a device, including tag 2, is passive and ‘listening’ until another device sends out a signal to initiate the process (ranging). When, for example, a tag 2 comes into reach and receives this ‘ping’ request, it can proceed with any one of applicable ranging schemes.

FIG. 6 shows a flowchart of a general method embodiment of the present invention. According to this embodiment of the present invention a method is provided for determining a hygiene compliance metric which indicates a usage of hygiene equipment, comprising a step S100 of detecting a usage instance indicating a use of distributed hygiene equipment by a user, the hygiene equipment arranged to dispense a hygiene consumable and/or to dispose of a hygiene consumable; a step S200 of transmitting radio signals from a tag. In the case that radio signals are at least received at the tag, this may be replaced by a step of receiving radio signals at that tag, and the signals are correspondingly at least transmitted from the positioning equipment. Following the example of the first variant, however, the method comprises a step S300 of determining information on a position of the tag by at least receiving radio signals from the tag, and determining information indicating a time of flight of a radio signal transmitted between the tag and the positioning equipment for determining the information on a position; receiving the information on a position of the tag; a step S400 of defining a zone into which the tag may enter and defining a rule; and a step S500 calculating the hygiene compliance metric based on the information on the position, the zone, and the rule.

It is noted that the above sequence can be modified and is not to be seen as requiring a specific order. For example, S400 can be carried out before any other steps or, in general, at least before performing step S500. Likewise, the order of information retrieval concerning the usage and opportunities may be reversed or the corresponding information can be obtained concurrently and continuously.

Although detailed embodiments have been described, these only serve to provide a better understanding of the invention and are not to be seen as limiting.

To this end, the embodiments described above are only descriptions of preferred embodiments of the present invention, and are not intended to limit the scope of the present invention. Various variations and modifications can be made to the technical solution of the present invention by those of ordinary skills in the art, without departing from the design and spirit of the present invention. The variations and modifications should all fall within the claim scope defined by the claims of the present invention.

What is claimed is:

1. A system for determining a hygiene compliance metric which indicates a usage of hygiene equipment, the system comprising:
distributed hygiene equipment arranged to dispense a hygiene consumable and/or to dispose of a hygiene consumable;
a tag configured to transmit and/or receive radio signals;
positioning equipment arranged to determine information on a position of the tag by receiving and/or transmitting radio signals from and/or to the tag, and configured to determine information indicating a time of flight of a radio signal transmitted between the tag and the positioning equipment for determining the information on the position; and
a processing entity which is configured to determine receive the information on the position of the tag, to define a zone into which the tag may enter, to define a rule, and to calculate the hygiene compliance metric based on the information on the position, the zone, and the rule.

2. The system of claim 1, wherein at least one of the positioning equipment and the tag is arranged to determine timing information indicating a timing when a radio signal was either transmitted or received by an antenna.

3. The system of claim 2, wherein at least one of the positioning equipment and the tag is arranged to generate a payload data based on the timing information and to transmit or to receive the payload data over a radio signal.

4. The system of claim 1, wherein the tag is arranged to be carried by a user and the processing entity is configured to process the information on the position of the tag in relation to a defined zone to thereby determine a usage instance of a piece of the distributed hygiene equipment or an opportunity in relation to the usage instance for, in turn, calculating the hygiene compliance metric.

5. The system of claim 1, wherein the tag is arranged to be associated with a piece of the distributed hygiene equipment and the processing entity is configured to define a zone in relation to a position of the tag, the zone being associated with a usage instance of the piece of the distributed hygiene equipment.

6. The system of claim 1, wherein the tag is arranged to be associated with an object or individual and the processing entity is configured to define a zone in relation to a position of the tag, the zone being associated with an opportunity in relation to a usage instance of a piece of distributed hygiene equipment.

7. The system of claim 1, wherein at least a piece of the distributed hygiene equipment is arranged to detect a usage instance indicating a use of the piece of the distributed hygiene equipment by a user, and to send information on the usage instance toward the processing entity.

8. The system of claim 1, wherein the processing entity is configured to process information on a type of event received as payload data from a radio signal from the tag, the type of event including an opportunity and/or a usage instance.

9. The system of claim 1, wherein the processing entity is configured to process data records comprising information on a type of usage and/or opportunity and information on an association to another record for determining the hygiene compliance metric.

10. The system of claim 1, wherein the processing entity is configured to consider a time between a determination of a usage instance and a determination of an opportunity for determining the hygiene compliance metric.

11. The system of claim 1, wherein the processing entity is configured to store information on several hygiene compliance indicators, each indicator relating to at least one determined usage instance and one determined opportunity.

12. The system of claim 1, wherein a piece of the distributed hygiene equipment is any one of a soap dispenser, a towel dispenser, a disinfectant dispenser, an alcogel dispenser, a tissue dispenser, a hygiene article dispenser, a waste bin, a used towel bin, and a toilet paper dispenser.

13. (canceled)

14. The system of claim 3, wherein the tag is arranged to be carried by a user and the processing entity is configured to process the information on the position of the tag in relation to a defined zone to thereby determine a usage instance of a piece of the distributed hygiene equipment or an opportunity in relation to the usage instance for, in turn, calculating the hygiene compliance metric,
wherein the tag is arranged to be associated with a piece of the distributed hygiene equipment and the processing entity is configured to define a zone in relation to a position of the tag, the zone being associated with the usage instance of the piece of the distributed hygiene equipment,
wherein the tag is arranged to be associated with an object or individual and the processing entity is configured to define a zone in relation to a position of the tag, the zone being associated with an opportunity in relation to the usage instance of the piece of distributed hygiene equipment,
wherein at least a piece of the distributed hygiene equipment is arranged to detect the usage instance indicating a use of the piece of the distributed hygiene equipment by a user, and to send information on the usage instance toward the processing entity,
wherein the processing entity is configured to process information on a type of event received as payload data from a radio signal from the tag, the type of event including an opportunity and/or the usage instance,
wherein the processing entity is configured to process data records comprising information on a type of usage and/or opportunity and information on an association to another record for determining the hygiene compliance metric,
wherein the processing entity is configured to consider a time between a determination of the usage instance and a determination of an opportunity for determining the hygiene compliance metric,
wherein the processing entity is configured to store information on several hygiene compliance indicators, each indicator relating to at least one determined usage instance and one determined opportunity, and
wherein a piece of the distributed hygiene equipment is any one of a soap dispenser, a towel dispenser, a disinfectant dispenser, an alcogel dispenser, a tissue dispenser, a hygiene article dispenser, a waste bin, a used towel bin, and a toilet paper dispenser.

15. A method for determining a hygiene compliance metric which indicates a usage of hygiene equipment, comprising:
detecting a usage instance of distributed hygiene equipment arranged to either dispense a hygiene consumable or to dispose of a hygiene consumable;
at least one of transmitting and receiving radio signal from or to a tag;
determine information on a position of the tag by receiving and/or transmitting radio signals from and/or to the tag and by determining information indicating a time of flight of a radio signal transmitted between the tag and a positioning equipment;
receiving the information on the position of the tag;
defining a zone into which the tag may enter;
defining a rule, and
calculating the hygiene compliance metric based on the information on the position, the zone, and the rule.
